(19) United States
(12) Patent Application Publication
(10) Pub. No.: US 2006/0101588 A1
(43) Pub. Date: May 18, 2006

(54) Washing machine with steam generating device and method for controlling the same

(75) Inventors: Jee Hun Park, Suwon-Si (KR); Hyung Gyoon Kim, Suwon-Si (KR); Sang Yeon Pyo, Suwon-Si (KR); Seon Woo Park, Suwon-Si (KR); Hyun Sook Kim, Suwon-Si (KR); Seong Min Oak, Masan-City (KR)

Correspondence Address: SUGHRUE MION, PLLC, 2100 PENNSYLVANIA AVENUE, N.W., SUITE 800, WASHINGTON, DC, 20037, US

(73) Assignee: SAMSUNG ELECTRONICS CO., LTD.

(21) Appl. No.: 11/153,346

(22) Filed: Jun. 16, 2005

(30) Foreign Application Priority Data
Nov. 16, 2004 (KR) 2004-93730

(51) Int. Cl. B08B 3/12; A01H 5/02; D06F 29/00; D06F 35/00

(52) U.S. Cl. 8/158; 8/147; 68/5 R; 68/20

(57) ABSTRACT

A washing machine having a drying function, which includes a steam generating device for spraying steam directly onto laundry, and a method for controlling the same. The method for controlling the washing machine, which supplies hot air, heated by a drying heater, to laundry through a drying duct, includes heating supplied wash water to generate steam; spraying the generated steam onto the laundry through the drying duct to perform a washing operation; determining whether or not a drying operation after the washing operation is performed; and performing a dehumidifying operation such that a drying fan is operated to remove moisture remaining in the drying duct when it is determined that the drying operation is not performed, thereby improving washing capacity, reducing an energy consumption rate, and effectively removing the moisture remaining in the drying duct when the drying operation after the washing operation is not performed.

### CROSS-REFERENCE TO RELATED APPLICATION

This application claims priority from Korean Patent Application No. 2004-93730, filed Nov. 16, 2004, in the Korean Intellectual Property Office, the entire disclosure of which is incorporated herein by reference.

### BACKGROUND OF THE INVENTION

1. Field of the Invention

Apparatuses and methods consistent with the present invention relate to a washing machine having a drying function, and more particularly, to a washing machine with a steam generating device for spraying steam directly onto laundry and a method for controlling the same.

2. Description of the Related Art

Generally, a washing machine having a drying function (conventionally, a drum washing machine) is an apparatus for drying laundry by supplying hot air, which is heated by a drying heater, by operation of a drying fan to the previously washed laundry. Thus, a drying operation is performed independently or in connection with a washing operation after a dehydrating operation.

Korean Patent Publication No. 10-0435241 discloses a drum washing machine having a drying function.

In the drum washing machine disclosed by the above Patent, when a drying operation is performed after a dehydrating operation is completed, a drying fan (blower) generates and sprays compressed air under the condition that a drying heater is operated to heat an inner space of a chamber.

The sprayed compressed air passes through the inner space of the chamber, thereby being changed into hot air. The hot air is supplied to laundry in a rotary drum, thereby drying moisture contained in the laundry.

The above conventional drum washing machine has high drying efficiency in the drying operation. However, since contaminants are removed from the laundry only by means of the dropping motion of the laundry and a detergent in the washing operation, the effectiveness of the washing operation in a conventional drum washing machine is limited.

In order to solve the above problem, a method for increasing a temperature of wash water using a washing heater installed at the lower portion of a tub has been proposed. However, since the wash water filling the tub must be heated to a temperature required to wash laundry, this method is disadvantageous in that an energy consumption rate is increased and an overall washing time is lengthened.

### SUMMARY OF THE INVENTION

Therefore, one aspect of the present invention is to provide a washing machine, having a drying function, which comprises a steam generating device, and a method for controlling the same, thereby improving washing capacity and reducing an energy consumption rate.

Another aspect of the present invention is to provide a washing machine comprising a steam generating device, which effectively removes moisture remaining in a drying duct when a drying operation is not performed after a washing operation is completed, and a method for controlling the same.

Yet another aspect of the present invention is to provide a washing machine comprising a steam generating device, which rapidly increases temperatures of wash water and laundry by supplying steam at a high temperature thereto, and a method for controlling the same, thereby shortening an overall washing time.

In accordance with one aspect, the present invention provides a method for controlling a washing machine, which supplies hot air, heated by a drying heater, to laundry through a drying duct, comprising: heating supplied wash water to generate steam; spraying the generated steam onto the laundry through the drying duct to perform a washing operation; determining whether or not a drying operation after the washing operation is performed; and performing a dehumidifying operation when it is determined that the drying operation is not performed.

In accordance with another aspect, the present invention provides a method for controlling a washing machine, which heats supplied wash water to generate steam, comprising spraying the generated steam onto laundry through a drying duct serving as a passage for supplying hot air to perform a washing operation; determining whether or not a drying operation after the washing operation is performed; and performing a dehumidifying operation when it is determined that the drying operation is not performed.

Additionally, the dehumidifying operation may be performed such that a drying fan is operated to remove moisture remaining in the drying duct.

In accordance with yet another aspect, the present invention provides a washing machine having a drying device for supplying hot air, heated by a drying heater, to laundry through a drying duct to dry the laundry, comprising a steam generating device connected to the drying duct for heating supplied wash water to generate steam.

Further, the steam generating device may include a steam heater for heating the supplied wash water to generate the steam of a high temperature; a steam supply pipe for supplying the steam generated from the steam heater; and a steam valve for controlling the supply of the wash water passing through the steam heater. Also, the steam supply pipe may be connected between the steam generating device and the drying duct.

Moreover, a spray nozzle for spraying the steam supplied from the steam supply pipe to the inside of the drying duct may be installed at an outlet of the steam supply pipe.

### BRIEF DESCRIPTION OF THE DRAWINGS

These and/or other aspects of the invention will become apparent and more readily appreciated from the following description of the exemplary embodiments, taken in conjunction with the accompanying drawings in which:

FIG. 1 is a perspective view of a washing machine according to an exemplary embodiment of the present invention;

FIG. 2 is a longitudinal sectional view of the washing machine according to an exemplary embodiment of the present invention;

FIG. 3 is a block diagram of a washing machine in accordance with an exemplary embodiment of the present invention; and

FIG. 4 is a flow chart illustrating a method for controlling a washing machine according to an exemplary embodiment of the present invention.

### DETAILED DESCRIPTION OF THE EXEMPLARY EMBODIMENTS

Reference will now be made in detail to the illustrative, non-limiting embodiments of the present invention, examples of which are illustrated in the accompanying drawings, wherein like reference numerals refer to like elements throughout. The exemplary embodiments are described below to explain aspects of the present invention by referring to the annexed drawings.

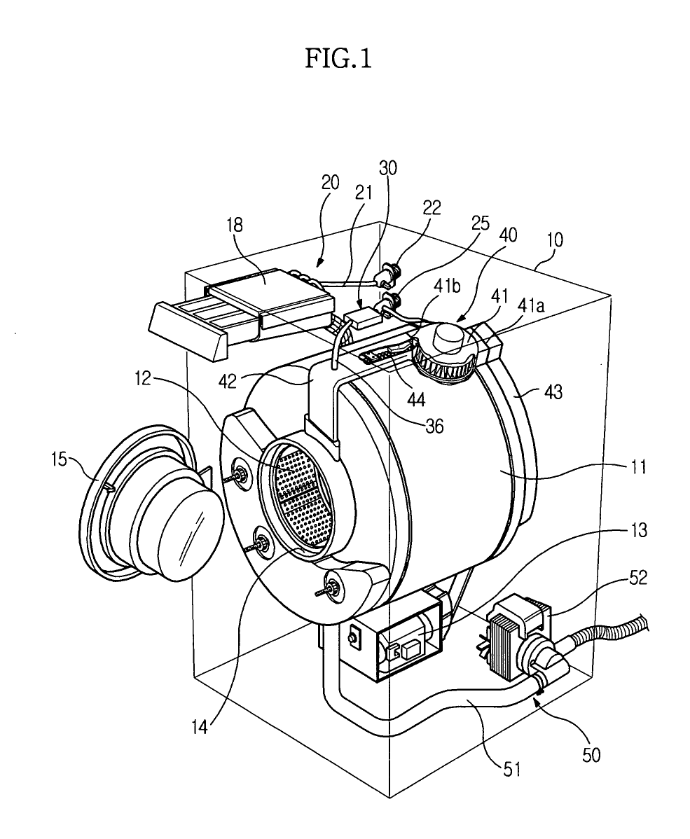

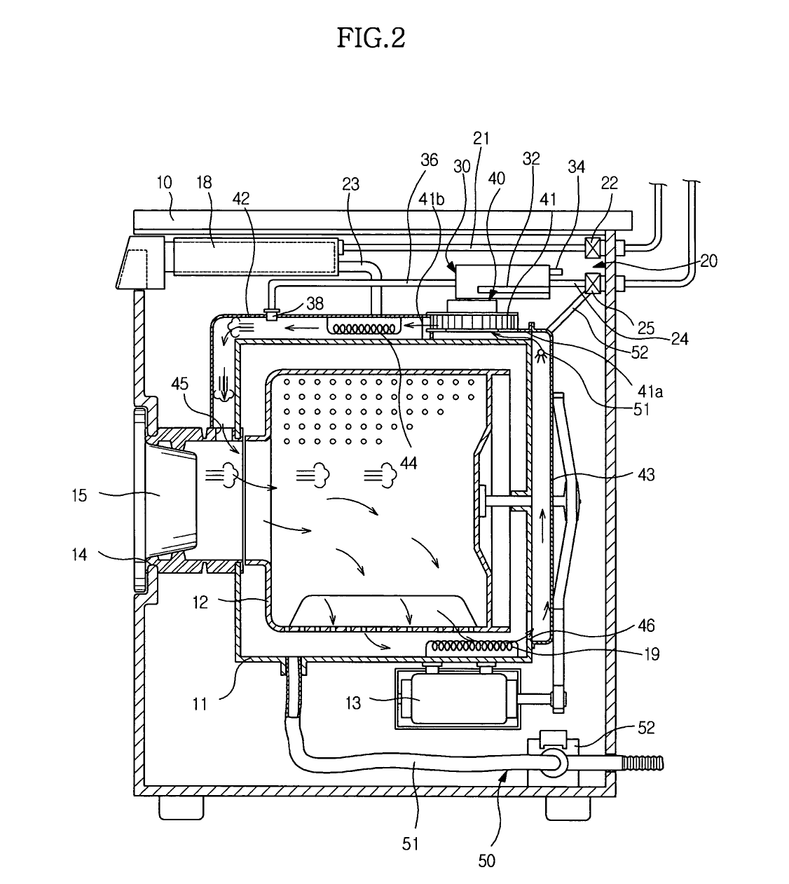

FIG. 1 is a perspective view of a washing machine according to an exemplary embodiment of the present invention, and FIG. 2 is a longitudinal sectional view of the washing machine according to an exemplary embodiment of the present invention.

As shown in FIGS. 1 and 2, the washing machine according to an exemplary embodiment of the present invention comprises a drum-shaped tub 11 installed in a main body 10 for containing wash water, and a drum 12 rotatably installed in the tub 11 and provided with a plurality of drain holes formed therethrough.

A motor 13 for rotating the drum 12 in a regular or reverse direction so as to perform a washing, rinsing, and dehydrating operations is installed below the tub 11, and a washing heater 19 for heating wash water supplied into the tub 11 when a user selects a temperature of the wash water is installed at a lower portion of the inside of the tub 11.

An opening 14 for taking laundry from the main body 10 is formed through front surfaces of the tub 11 and the drum 12, and a door 15 for opening and closing the opening 14 is installed at the front surface of the main body 10.

A detergent supply device 18 for supplying a detergent to the inside of the tub 11, a steam generating device 30 for supplying steam to the inside of the tub 11, and a water supply device 20 for supplying water to the tub 11 and the steam generating device 30 are installed above the tub 11.

The detergent supply device 18 includes a space for containing the detergent therein, and is installed close to the front surface of the main body 10 so that a user can easily put the detergent into the detergent supply device 18.

The water supply device 20 includes a water supply pipe 21 for supplying water to the inside of the tub 11, and a water supply valve 22 installed in the water supply pipe 21 for controlling the supply of the water to the water supply pipe 21. Here, the water supply pipe 21 is connected to the detergent supply device 18 so that the water supplied from the outside is supplied to the detergent supply device 18. A connection pipe 23 is installed between the detergent supply device 18 and the tub 11 such that the water having passed through the detergent supply device 18 is supplied to the tub 11. The connection pipe 23 causes water, which is supplied to the inside of the tub 11, to pass through the detergent supply device 18, thereby allowing the detergent contained in the detergent supply device 18 to be dissolved in the water and then supplied to the inside of the tub 11.

The water supply device 20 further includes a steaming water supply pipe 24 for supplying water to the steam generating device 30 as well as to the detergent supply device 18, and a steam valve 25 installed in the steaming water supply pipe 24 for controlling the supply of the water to the steam generating device 30.

Although the water supply device 20 includes the water supply valve 22 and the steam valve 25, which are installed separately as described above, the present invention is not limited thereto, but the water supply device 20 may include an integrated assembly of the water supply valve 22 and the steam valve 25 using a conventional electric three-way or four-way valve.

The steam generating device 30 includes a U-shaped steam heater 32 installed therein for rapidly heating water passing therethrough to generate steam at a high temperature of at least 1 00° C, a temperature sensor 34 installed at one side of the steam generating device 30 for sensing the temperature of the steam generating device 30, a steam supply pipe 36 extended from the steam generating device 30 to a drying duct 42, and a discharge nozzle 38 installed at an outlet of the steam supply pipe 36.

Although the steam heater 32 is installed in the steam generating device 30, as described above, the present invention is not limited thereto, but the steam heater 32 may be an external heater having a structure contacting the external surface of an upper or lower portion of the steam generating device 30 or surrounding the external circumferential surface of the steam generating device 30.

The washing machine of the present invention further comprises a drying device 40 for drying laundry after the laundry is dehydrated. The drying device 40 includes a drying fan 41 installed on the upper surface of the tub 11, a drying duct 42 for connecting an outlet 41b of the drying fan 41 and an air inlet 45 formed through the upper portion of the opening 14 of the tub 11, and a condensing duct 43 installed at the rear portion of the tub 11 for connecting an air outlet 46 formed through the rear portion of the tub 11 and an inlet 41a of the drying fan 41.

The drying device 40 further includes a drying heater 44 installed in the drying duct for supplying hot air to the inside of the tub 11, and a condensing device (not shown) installed in the condensing duct 43 for condensing moisture and removing the moisture when moisture steam generated by drying the laundry passes through the condensing duct 43. The above configuration of the drying device 40 serves to heat air, blown by the operation of the drying fan 41, using the drying heater 44 and then to supply the heated air to the inside of the tub 11, thereby heating and drying the laundry in the tub 11. Further, the above configuration of the drying device 40 serves to remove moisture from the air when the moisture steam generated by drying the laundry is sucked into the drying fan 41 through the condensing duct 43.

The condensing device includes a cooling water spray nozzle 53 installed at an upper portion of the inside of the condensing duct 43 for spraying cooling water to the inside of the condensing duct 43, and a cooling water supply pipe 54 connected to the water supply device 20 for supplying the cooling water to the cooling water spray nozzle 53. The above configuration of the condensing device serves to cause the cooling water, sprayed from the cooling water spray nozzle 53, to flow down along the inner surface of the condensing duct 43 so that a contact surface between moisture ascending from the lower part and the cooling water is increased, thereby improving dehumidifying effects.

The washing machine according to an exemplary embodiment of the present invention further comprises a drain device 50 for discharging the water of the inside of the tub 11. The drain device 50 includes a drain pipe 51 connected to the lower portion of the tub 11 so as to guide the water of the tub 11 to the outside, and a drain pump 52 installed in the drain pipe 51.

Hereinafter, the operation of the above-described washing machine will be described.

When the washing machine is operated under the condition that laundry is put into the drum 12 and a detergent is put into the detergent supply device 18, the water supply valve 22 of the water supply device 20 is opened so that wash water is supplied to the detergent supply device 18. The detergent in the detergent supply device 18 is dissolved in the wash water. Then, the wash water and the dissolved detergent are supplied to the inside of the tub 11.

After the supply of the wash water to the inside of the drum 12 is completed, the washing heater 19 is operated to heat the wash water in the tub 11. After a designated time elapses and the wash water is heated to a temperature required to wash the laundry, the drum 12 is rotated in a regular or reverse direction by the operation of the motor 13.

Here, the laundry is moved by lifters formed along the inner surface of the drum 12 so that the laundry is washed by the friction with the drum 12 and the degradability of the detergent.

In the case where a user selects a steam course, the steam valve 25 is opened under the condition that the wash water is heated by the washing heater 19, thereby allowing the wash water to be supplied to the steam generating device 30 through the steaming water supply pipe 24. Thus, wash water, which is supplied to the steam generating device 30, is rapidly heated by the steam heater 32, thereby generating steam at a high temperature of at least 100° C.

The steam at a high temperature is supplied to the inside of the tub 11 through the steam supply pipe 36 and the drying duct 42, and is sprayed onto the wash water and the laundry in the tub 11. Further, the wash water is heated by the washing heater 19.

Accordingly, the temperature of the laundry, which is sufficiently soaked by the wash water containing the detergent, i.e., a detergent solution, is rapidly increased by the steam at high temperature so that the detergent solution soaking the laundry is in an activated state. Further, when a designated time elapses and the supply of the steam is stopped, the drum 12 is rotated by the operation of the motor 13, thereby rapidly removing contaminants from the laundry by means of the friction between the laundry and the drum 12 and the activated detergent solution so as to improve washing performance.

After the washing operation is completed, a rinsing operation in which dehydration and repeated supply of the wash water occurs, is performed. During the rinsing operation, the water supply valve 22 is opened so that water is supplied to the inside of the tub 11 through the water supply pipe 21. The drain pump 52 is operated so that the water in the tub 11 is discharged to the outside through the drain pipe 51.

When the final dehydrating operation is performed after the rinsing operation is completed, the drain pump 52 is operated, and the drum 12 is rotated at a high speed for a designated time, thereby dehydrating the laundry.

When a drying operation is performed under the condition that the dehydration of the laundry is completed as described above, the drum 12 is slowly rotated by the operation of the motor 13 so that the laundry elevated in the drum 12 is dropped. When the drying fan 41 is operated under the above state, air in the tub 11 is repeatedly circulated such that the air is sucked into the drying fan 41 through the condensing duct 43, and is then discharged to the inside of the tub 11 through the drying duct 42.

Here, the air in the drying duct 42, which is heated by the drying heater 44, flows into the drum 12, and the laundry in the drum 12 is heated and dried by the hot air. When moisture steam generated from the drying process flows into the condensing duct 43 and is circulated into the drying duct 41, moisture in the steam is condensed and eliminated by the condensing device in the condensing duct 43.

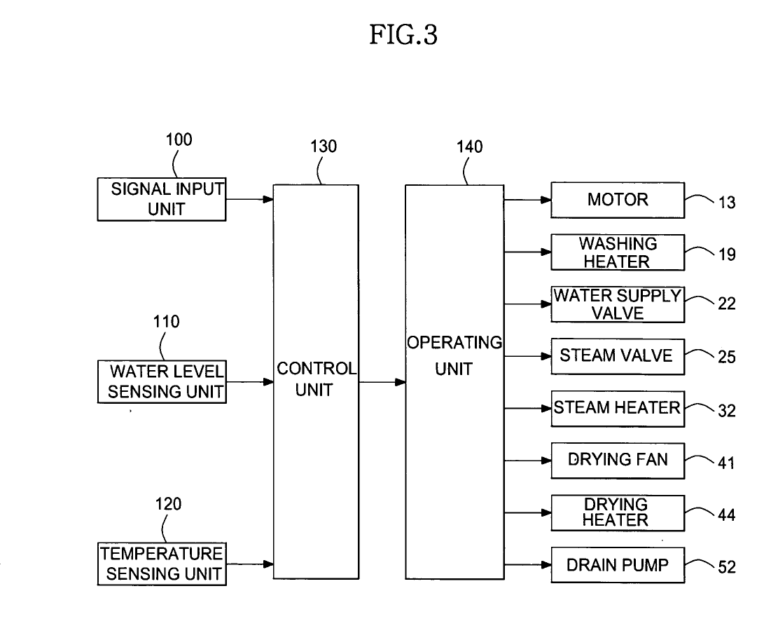

FIG. 3 is a block diagram of a washing machine in accordance with an exemplary embodiment of the present invention. The washing machine comprises a signal input unit 100, a water level sensing unit 110, a temperature sensing unit 120, a control unit 130, and an operating unit 140.

The signal input unit 100 allows a user to input selected operating data, such as a washing course, a washing temperature, a dehydrating rotation speed, and an additional rinsing operation, according to material types of the laundry to the control unit 130. The water level sensing unit 110 senses the level of the wash water supplied to the inside of the tub 11. The temperature sensing unit 120 includes a temperature sensor 34 for sensing the temperature of the wash water supplied to the tub 11 and the temperature of the steam generating device 30.

The control unit 130 is a microcomputer for controlling the washing machine according to the operating data inputted from the signal input unit 100. The control unit 130 controls operation of the drying fan 41 so that moisture remaining in the drying duct 42 or the tub 11 is removed when the drying operation is not performed after the washing operation is completed.

The operating unit 140 operates the motor 13, the washing heater 19, the water supply valve 22, the steam valve 25, the steam heater 32, the drying fan 41, the drying heater 44, and the drain pump 52 based on an operation control signal of the control unit 130.

Hereinafter, the operation and effects of the above-described washing machine and a method for controlling the same will be described in detail.

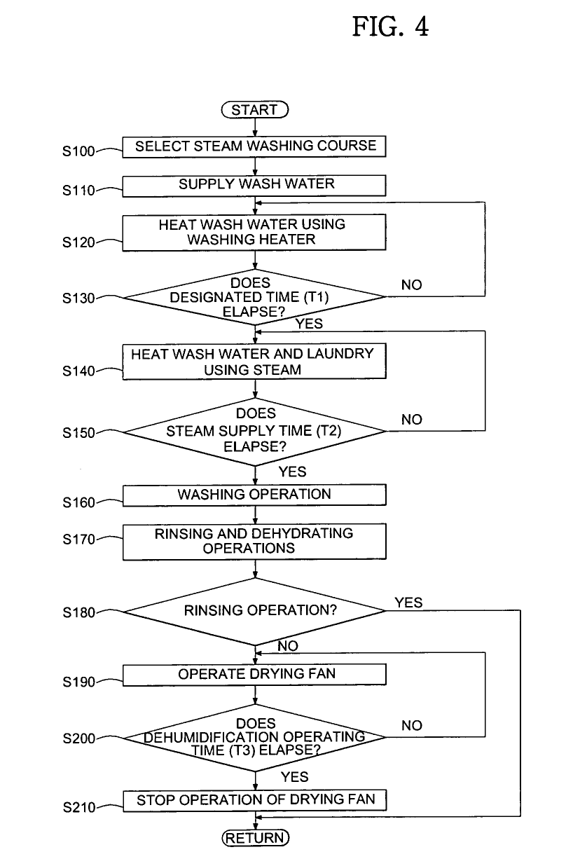

FIG. 4 is a flow chart illustrating a method for controlling a washing machine of the present invention. In FIG. 4, “S” denotes an operation.

The method for controlling the washing machine according to an exemplary embodiment of the present invention comprises operations (S100˜S110) of supplying wash water to the inside of the tub 11 according to a steam washing course selected by a user; operations (S120˜S130) of heating the wash water supplied to the inside of the tub 11 using the washing heater 19; operations (S140˜S150) of activating a detergent solution soaking laundry by spraying steam of a high temperature of at least 100 to the wash water and the laundry in the tub 11 heated by the washing heater 19; an operation (S160) of performing a washing operation using the washing heater 19 and the steam according to a washing course selected by the user; an operation (S170) of performing rinsing and dehydrating operations after the washing operation is completed; and operations (S180˜S210) of removing moisture remaining in the drying duct 42 or the tub 11 when a drying operation is not performed after the washing operation is completed.

Now, the operation of the method for controlling the above-described washing machine will be described in more detail.

When the user selects operating data, such as a washing course (for example, a steam washing course), a washing temperature, a dehydrating rotation speed, and an additional rinsing operation, the operating data selected by the user are inputted to the control unit 130 through the signal input unit 100 (S100).

Then, the control unit 130 turns the water supply valve 22 on so that wash water is supplied to the detergent supply device 18, and a detergent in the detergent supply device 18 is dissolved in the wash water. Next, the wash water and the dissolved detergent are supplied to the tub 11 (S110).

After a designated quantity of the wash water is supplied to the inside of the tub 11 through the water supply pipe 21, the control unit 130 turns the water supply valve 22 off so that the supply of the wash water into the water supply pipe 21 is stopped.

After the supply of the wash water is completed, the washing heater 19 is operated by the controlling operation of the control unit 130, thereby heating the wash water filling the tub 11 (S120). An operating time of the washing heater 19 is checked, and it is determined whether or not a designated time (T1; a time taken to heat the wash water to a temperature required to perform the washing operation using the washing heater 19) elapses (S130). When it is determined that the designated time (T1) has elapsed, the operation of the washing heater 19 is stopped.

Thereafter, the control unit 130 controls the steam valve 25 so that the wash water is supplied to the steam generating device 30 through the steaming water supply pipe 24, and the wash water supplied to the steam generating device 30 is rapidly heated by the steam heater 32 to generate steam of a high temperature of at least 100° C.

The steam of the high temperature is supplied to the inside of the tub 11 through the steam supply pipe 36 and the drying duct 42, and additionally heats the wash water and the laundry in the tub 11, which were previously heated by the washing heater 19 (S140).

Since the method for controlling the washing machine according to an exemplary embodiment of the present invention heats the wash water using the washing heater 19 and supplies a large quantity of the steam at a high temperature of at least 100° C. to the inside of the tub 11 through the steam generating device 30, the method provides a rapid increase in the temperature of the wash water as compared to the conventional method, in which the wash water is heated only by the washing heater 19 installed in the lower portion of the tub 11. Accordingly, overall washing time is remarkably shortened and the quantity of energy consumed to heat the wash water is reduced.

Further, since the method for controlling the washing machine in accordance with an exemplary embodiment of the present invention sprays the steam at a high temperature onto the laundry, which is sufficiently soaked by the detergent solution, the temperature of the laundry is rapidly increased. Thus, the washing time is shortened and the detergency of the detergent solution in the activated state contained in the laundry is improved so as to increase the washing effects of the washing machine.

A steam supply time of the steam generating device 30 is checked, and it is determined whether or not a designated time (T2; overall operating time of the steam generating device 30 from a point of the time when the steam heater 32 is preheated, approximately 6˜10 minutes) elapses (S150). When it is determined that the designated time (T2) has elapsed, the operation of the steam heater 32 is stopped so that the supply of the steam is stopped.

When the heating of the wash water using the washing heater 19 and the supply of the steam are stopped, the drum 12 is rotated by the operation of the motor 13 so that contaminants are rapidly removed from the laundry by means of the friction between the laundry and the drum 12 and the activated detergent solution, thereby performing the washing operation having an improved washing performance (S160).

When the washing operation is completed, a rinsing operation, in which dehydration and repeated supply of the wash water occurs, is performed according to the selected operating data, and, when the final dehydrating operation is performed after the rinsing operation is completed, a dehydrating operation, in which the drum 12 is rotated at a high speed for a designated time so that the laundry is dehydrated, is performed (S170).

Since the washing machine according to an exemplary embodiment of the present invention has a structure such that the steam generated from the steam generating device 30 is supplied to the inside of the tub 11 through the steam supply pipe 36 and the drying duct 42, moisture remains in the steam supply pipe 36 and the drying duct 42 due to the condensation.

Also, the moisture remaining in the steam supply pipe 36, which is made of a rubber hose, does not damage the steam supply pipe 36.

When a drying operation is performed under the condition that the dehydration of the laundry is completed, the moisture remaining in the drying duct 42 is completely removed, thereby not damaging the drying heater 44. On the other hand, when the drying operation is not performed after the washing operation is completed, a dehumidifying operation for removing the moisture remaining in the drying duct 42 or the tub 11 is performed.

Accordingly, the control unit 130 determines whether or not the drying operation after the dehydrating operation is performed (S180). When it is determined that the drying operation is not performed, the control unit 130 operates the drying fan 41 so that the dehumidifying operation for removing the moisture remaining in the drying duct 42 or the tub 11 is performed.

The control unit 130 checks the operating time of the drying fan 41, and determines whether or not a dehumidification operating time (T3; a time taken to remove the moisture remaining in the drying duct 42 or the tub 11) elapses (S200). When it is determined that the designated time (T3) has elapsed, the control unit 130 stops the operation of the drying fan 41 so that the dehumidifying operation is stopped (S210).

Although exemplary embodiments of the present invention describe the drum washing machine having a drying function, the present invention is not limited thereto, but may be applied to any washing or drying machine, which can supply hot air.

As apparent from the above description, aspects of the present invention provide a washing machine having a drying function, which comprises a steam generating device installed therein for spraying steam directly onto laundry without modifying a mold, and a method for controlling the same, thereby improving washing efficiency and reducing an energy consumption rate.

Further, since moisture remaining in a drying duct is effectively removed when a drying operation after the washing operation is not performed, the washing machine and the method for controlling the same according to aspects of the present invention prevent a heater installed in the drying duct from being damaged.

Moreover, since steam of a high temperature is supplied to rapidly increase the temperature of wash water and the laundry, the washing machine and the method for controlling the same according to aspects of the present invention shorten an overall washing time and eliminate wrinkling of the laundry as a result of the use of steam.

Although exemplary embodiments of the invention has been shown and described, it would be appreciated by those skilled in the art that changes may be made without departing from the principles and spirit of the invention, the scope of which is defined in the claims and their equivalents.

1. A method for controlling a washing machine, which supplies hot air, heated by a drying heater, to laundry through a drying duct, comprising:
heating wash water to generate steam; and
spraying the generated steam onto the laundry through the drying duct to perform a washing operation.

2. The method as set forth in claim 1, further comprising:
determining whether or not a drying operation after the washing operation is performed; and
performing a dehumidifying operation when it is determined that the drying operation is not performed.

3. The method as set forth in claim 2,
wherein the dehumidifying operation is performed such that a drying fan is operated to remove moisture remaining in the drying duct.

4. A method for controlling a washing machine, which heats wash water to generate steam, comprising spraying the generated steam onto laundry through a drying duct serving as a passage for supplying hot air to perform a washing operation.

5. The method as set forth in claim 4, further comprising:
determining whether or not a drying operation after the washing operation is performed; and
performing a dehumidifying operation when it is determined that the drying operation is not performed.

6. The method as set forth in claim 5,
wherein the dehumidifying operation is performed such that a drying fan is operated to remove moisture remaining in the drying duct.

7. A washing machine having a drying device for supplying hot air, heated by a drying heater, to laundry through a drying duct to dry the laundry, comprising a steam generating device connected to the drying duct for heating wash water to generate steam.

8. The washing machine as set forth in claim 7, wherein the steam generating device includes:
a steam heater for heating the wash water to generate the steam at a high temperature;
a steam supply pipe for supplying the steam which is generated from the steam heater; and
a steam valve for controlling the supply of the wash water passing through the steam heater.

9. The washing machine as set forth in claim 8,
wherein the steam supply pipe is connected between the steam generating device and the drying duct.

10. The washing machine as set forth in claim 8,
wherein a spray nozzle for spraying steam which is supplied from the steam supply pipe to inside of the drying duct is installed at an outlet of the steam supply pipe.

11. A method for controlling a washing machine, which performs a washing operation and a drying operation, comprising:
determining whether or not the drying operation is performed after the washing operation; and
performing the drying operation when it is determined that the drying operation is not performed after the washing operation.

12. The method as set forth in claim 11, wherein the drying operation is performed such that drying air is supplied to the inside of the washing machine to remove moisture in the washing machine.

13. The method as set forth in claim 12, wherein, in the drying operation, the drying air is heated to hot air and the hot air is supplied to the inside of the washing machine to remove moisture remaining in the washing machine.

14. A washing machine comprising:
a main body comprising a tub;
a drying device comprising a drying fan and a drying duct; and
a control unit for controlling a washing operation and a drying operation,
wherein the control unit determines whether or not the drying operation is performed after the washing operation, and performs the drying operation, when it is determined that the drying operation is not performed after the washing operation, so that a dehumidifying operation for removing moisture remaining in the washing machine is performed.

15. The washing machine as set forth in claim 14, wherein the dehumidifying operation is performed such that a drying fan is operated to remove moisture remaining in the drying duct or the tub.

16. The washing machine as set forth in claim 15, wherein:
the drying device further comprises a drying heater for supplying hot air; and
in the dehumidifying operation, the hot air supplied by the drying heater is supplied to the drying duct or the tub.
